(19) United States
(12) Patent Application Publication
(10) Pub. No.: US 2022/0370704 A1
(43) Pub. Date: Nov. 24, 2022

(54) VACUUM SEALED BANDAGE AND METHODS OF USE

(71) Applicant: Stacey Goller, Manahawkin, NJ (US)

(72) Inventor: Stacey Goller, Manahawkin, NJ (US)

(21) Appl. No.: 17/323,215

(22) Filed: May 18, 2021

(51) Int. Cl. A61M 1/00; A61F 13/02

(52) U.S. Cl. CPC A61M 1/91; A61F 13/0216; A61F 2013/00604

(57) ABSTRACT

The present disclosure relates to wound bandages. More specifically, the present disclosure describes a vacuum sealed bandage having a film layer that is manipulable into a wound by way of negative pressure imparted by a removable medical syringe. The vacuum sealed bandage of the present disclosure does not have a vacuum pump, does not constantly apply negative pressure to the wound through an electro-mechanical device or vacuum pump, and/or does not have chords or tubes positioned to opposite sides of the film layer or extending from one side of the film layer to another side of a film layer where the chord or tube is positioned between the film and the wound.

### BACKGROUND

This disclosure relates to bandages and, more specifically, a vacuum sealed closure that is also a bandage for applying negative pressure to a wound for improved healing of the wound.

Vacuum sealed closures have been relied on to provide negative pressure to a wound to remove air from between a closure and the wound, remove fluids from the wound, and even remove bacteria from the wound in order to reduce swelling and infection. Such techniques are referred to as vacuum-assisted closures (VAC) or VAC therapy and more generally referred to as wound VACs. A wound VAC device relies on a vacuum pump connected to a tube which is inserted through an opening of a closure or from under an closure such that it is positioned between the closure and the wound. The tube is positioned between the closure and the wound in order to remove air or fluids at, or around, the wound from between the closure and the wound. The vacuum pump applies negative pressure between the closure and the wound to apply pressure to remove the air and fluids at, or around, the wound. By applying negative pressure, the enclosure is pulled toward the wound. A dressing may additionally be provided between the enclosure and the wound.

VAC devices must remain under a constant vacuum and, therefore, requires the vacuum pump to be in constant operation. If the vacuum pump ceases to operate, or is removed from the closure, the vacuum, or negative pressure, between the closure and the wound is not maintained. VAC devices are administered and monitored by medical professionals, or require training to operate, as the vacuum pump remains in operation during use and is a form of therapy. Often the VAC device must be removed periodically to change a dressing positioned between the enclosure and the wound and the VAC device is reapplied to continue the therapy or under repetitive usage.

VAC devices are cumbersome, incorporate numerous components, are electro-mechanical devices requiring a power supply, require extensive training for use, and require constant operation of a vacuum pump to maintain pressure at a wound. This limits patient access to VAC devices and, more specifically, utilization of a VAC device in emergency and/or remote environments. What is needed is a device that is additionally a bandage, that applies negative pressure to a wound without utilizing an electro-mechanical device requiring a power supply, or vacuum pump, or without requiring the constant pressure being applied through an electro-mechanical device, or vacuum pump, and that is of a compact design. Moreover, what is needed is a vacuum sealed bandage that may be provided in a single sterilized package without reliance on, or constant utilization of, an electro-mechanical device or power supply, such as a vacuum pump.

### SUMMARY

The present disclosure relates to a vacuum sealed bandage as an alternative to a VAC device.

In one example, a vacuum sealed bandage comprises a first film material and a second film material. The first film material has adhesive on one side configured to be applied to skin and to adhere the first film material in an airtight arrangement with the skin. The second film material extends from the first film material. A self-sealing valve further extends from, and is sealingly attached to, the second film material. The first film material, the second film material, and the self-sealing valve form a contiguous vacuum barrier when the self-sealing valve is closed. The second film material is manipulable into multiple positions or arrangements. In some examples, the first film material forms a perimeter about the second film material. The second film material may be loosely configured to be moved from one side of the first film material to the opposite side of the first film material through the perimeter formed by the first film material. The self-sealing valve may extend from a first side of the vacuum barrier and may be flush on the second side of the vacuum barrier. The second film material may extend from a side of the first film material in the same direction as the self-sealing valve extends from the first side of the vacuum barrier. In some examples the second film material extends from a side of the first film material in a direction opposite the side having the adhesive and the second film material may extend in a twisted arrangement. The loose arrangement of the second film material may advance to and from the first film material. In some examples, the second film material may be folded upon itself. In some examples, the first film material may be more rigid than the second film material. In some examples, the first film material may be layered and the second film material may extend from and between the layers of the first film material. In some examples the first film material and the second film material are layered and are positioned in an overlapping arrangement relative one another. The self-sealing valve may be threaded to receive a removable medical syringe. A removable medical syringe may be inserted into the self-sealing valve and may, additionally, hold the self-sealing valve open. The removable medical syringe may be sealably connected to the self-sealing valve.

In operation, the removable medical syringe may create a negative pressure between a wound and the vacuum barrier when the first film material is in the airtight arrangement with the skin. The removable medical syringe may adjust the amount of negative pressure between the wound and the vacuum barrier. The negative pressure may manipulate the second film material into the wound. The negative pressure is relied on to adhere the second film material to the wound. Additionally, or alternatively, the negative pressure may tunnel the second film material into the wound. Negative pressure may be maintained between the second film material and the wound by way of the self-sealing valve. More specifically, the negative pressure may be maintained between the second film material and the wound with the syringe removed from the self-sealing valve. Negative pressure is maintained between the second film material and the wound without a vacuum pump and/or does not require a power supply for operation. In other words, the vacuum sealed bandage does not have a vacuum pump and/or does not require a power supply for operation. A tube is additionally not required to extend from one side of the vacuum barrier to the opposite side of the vacuum barrier, such as would be required by a wound VAC device.

A method for applying a vacuum sealed bandage to a wound is also recited herein. The method comprises the steps of:
adhering a first film material having an adhesive on one side to a skin surface forming an airtight arrangement with the skin surface;
sealingly attaching a removable medical syringe to a self-sealing valve where the self-sealing valve is sealingly attached to a second film material that extends from the first film material where the first film material, the second film material, and the self-sealing valve form a contiguous vacuum barrier when the self-sealing valve is closed;
creating negative pressure between the second film material and a wound in the skin by drawing air or fluid from between the contiguous vacuum barrier and the wound into the removable medical syringe;
manipulating the second film material into the wound by way of drawing air or fluid from between the contiguous vacuum barrier and the wound; and
removing the removable medical syringe from the self-sealing valve where the self-sealing valve seals upon removal of the removable medical syringe.

The method for applying a vacuum sealed bandage to a wound may further comprise the steps of:
untwisting the second film material when manipulating the second film material into the wound wherein the second film material is in a twisted arrangement before the step of adhering;
moving the second film material from one side of the first film material to the opposite side of the first film material having the adhesive when manipulating the second film material into the wound;
adjusting the amount of negative pressure between the second film material and a wound by adjusting the removable medical syringe;
maintaining the negative pressure created between the second film material and the wound upon removing the removable medical syringe; and/or
tunneling the second film material into the wound.

In examples of the method for applying a vacuum sealed bandage to a wound the step of sealingly attaching the removable medical syringe to the self-sealing valve includes threading the removable medical syringe to the self-sealing valve. In some examples of the method for applying a vacuum sealed bandage to a wound the method is performed without a vacuum pump and/or a power supply. In some examples, the method is performed without a tube inserted from one side of the vacuum barrier to the opposite side of the vacuum barrier such that it is positioned between the vacuum barrier and the wound.

The method for applying a vacuum sealed bandage to the wound may further comprise the steps of:
adhering a first film material of a second vacuum sealed bandage to a skin surface where the second vacuum sealed bandage is larger than and overtop a first vacuum sealed bandage as described above and the first film material of the second vacuum sealed bandage has an adhesive on one side forming an airtight arrangement with the skin;
sealingly attaching a removable medical syringe to a self-sealing valve of the second vacuum sealed bandage where the self-sealing valve is sealingly attached to a second film material of the second vacuum sealed bandage that extends from the first film material of the second vacuum sealed bandage where the first film material, the second film material, and the self-sealing valve of the second sealed bandage form a contiguous vacuum barrier when the self-sealing valve is closed;
creating negative pressure between the second film material of the second vacuum sealed bandage and a first vacuum sealed bandage by drawing air or fluid from between the contiguous vacuum barrier of the second vacuum sealed bandage and the first vacuum sealed bandage into the removable medical syringe;
manipulating the second film material of the second vacuum sealed bandage into the first vacuum sealed bandage and the wound by way of drawing air or fluid from between the contiguous vacuum barrier of the second sealed bandage and the first vacuum sealed bandage; and
removing the removable medical syringe from the self-sealing valve of the second vacuum sealed bandage where the self-sealing valve seals upon removal of the removable medical syringe.

Also disclosed herein is a packaged vacuum sealed bandage system. An example of a packaged sealed bandage may comprise, consist essentially of, or consist of a sterile wrapping and a vacuum sealed bandage sealed within the sterile wrapping. The vacuum sealed bandage may have a first film material having adhesive on one side configured to be applied to skin to adhere to the first film material in an airtight arrangement with the skin. A second film extends from the first film. A self-sealing valve extends from and sealingly attaches to the second film material. The first film material, the second film material, and the self-sealing valve form a contiguous vacuum barrier when the self-sealing valve is closed and the second film material is manipulated into multiple positions or arrangements. In some examples, the packaged vacuum sealed bandage system may further comprise, consistent essentially of, or consist of a removable medical syringe additionally sealed within the sterile wrapping. In some examples the second film material is in a twisted arrangement. In some examples, the second film material extends to one side of the first film material opposite the side of the first film material having adhesive. In examples, the packaged vacuum sealed bandage is without a power supply and is not for use with a power supply. In examples, the packaged vacuum sealed bandage is without a vacuum pump and not for use with a tube and not for use with a vacuum pump.

The foregoing and other objects, features, and advantages of the examples will be apparent from the following more detailed descriptions of particular examples as illustrated in the accompanying drawings wherein like reference numbers represent like parts of the examples.

### BRIEF DESCRIPTION OF THE DRAWINGS

Reference is made to the accompanying drawings in which particular examples and further benefits of the examples are illustrated as described in more detail in the description below, in which:

FIG. 1 is a top side perspective view of a vacuum sealed bandage, in accordance with an example of the disclosure.

FIG. 2 is a top side view of a vacuum sealed bandage, in accordance with an example of the disclosure.

FIG. 3 is a bottom view of a vacuum sealed bandage, in accordance with an example of the disclosure.

FIG. 4 is a side view of a vacuum sealed bandage, in accordance with an example of the disclosure.

FIG. 5 is a cross-section of a vacuum sealed bandage taken at line 5-5 of FIG. 2, in accordance with an example of the disclosure.

FIG. 6 is a top side perspective view of a vacuum sealed bandage with a removable medical syringe, in accordance with an example of the disclosure.

FIG. 7 is a top side perspective view of a vacuum sealed bandage with the second film material manipulated, in accordance with an example of the disclosure.

FIG. 8 is a bottom side perspective view of a vacuum sealed bandage with the second film material manipulated, in accordance with an example of the disclosure.

FIG. 9 is a top side perspective view of a vacuum sealed bandage within a packaged vacuum sealed bandage system, in accordance with an example of the disclosure.

FIG. 10 is a top side perspective view of a vacuum sealed bandage within a packaged vacuum sealed bandage system with a removable medical syringe, in accordance with an example of the disclosure.

FIG. 11 is a top side perspective view of a valve arrangement for use with a vacuum sealed bandage system, in accordance with an example of the disclosure.

FIG. 12A is a cross-section of the valve arrangement of FIG. 11 with the valve closed, in accordance with an example of the disclosure.

FIG. 12B is a cross-section of the valve arrangement of FIG. 11 with the valve open, in accordance with an example of the disclosure.

### DETAILED DESCRIPTION

VAC devices relied on for VAC therapy have become a valuable tool for wound care. VAC devices, however, are cumbersome, require significant training for use, require utilization of an electro-mechanical device, or vacuum pump, require a power supply, and require constant application of the electro-mechanical device, or vacuum pump, in order to maintain negative pressure on a wound. As used herein a power supply is a device that supplies electrical power, such as, for example, supplying electrical power directly from a fuel cell, generator, alternator, power source, power grid, outlet, chord, energy storage device, or battery. Because VAC devices are relied on by professionals in hospitals or medical facilities, VAC devices are typically not available in emergency situations, in the field, or for medical care in remote environments. The present disclosure is directed to a vacuum sealed bandage that is an alternative to the above-described VAC devices. The vacuum sealed bandage of the present disclosure comes in a compact design, may be packaged in a sterile and/or compact packaging for individual use, does not require an electro-mechanical device such as a vacuum pump, does not require a power supply, and, more specifically, does not require the constant operation of an electro-mechanical device or vacuum pump to maintain negative pressure on a wound. The vacuum sealed bandage of the present disclosure may be provided and/or utilized in emergency situations, in the field, in remote environments, in military environments, and/or in first aid kits. Additionally, the vacuum sealed bandage of the present disclosure may be mass produced, easily stored, easily transported for added mobility, and easily utilized or shipped without needing a power supply or requiring maintenance of an electro-mechanical device, or vacuum pump, for its utilization, thereby, providing more efficient and improved patient accessibility.

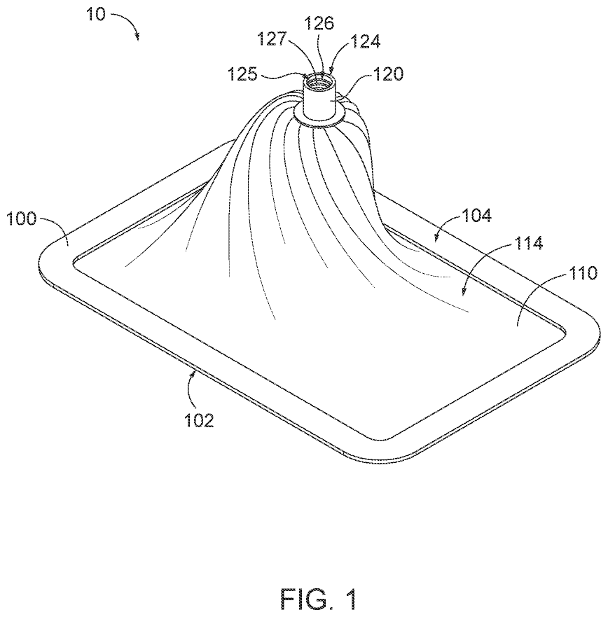

FIG. 1 illustrates a top side perspective view of a vacuum sealed bandage 10 of the present disclosure for covering a wound. The vacuum sealed bandage 10 has a first film material 100, the first film material 100 may form a perimeter about a second film material 110. The first film material 100 has adhesive 106 on a bottom side 102 as illustrated by FIG. 3. The adhesive 106 is configured to adhere the first film material 100 to a patient's skin, or body, in a sealed or airtight arrangement. The adhesive 106 sticks to the skin, or body, maintaining the bottom side of the first film material in contact with the skin and forming an airtight, or sealed, arrangement between the first film material 100 and the skin, or body.

A second film material 110 extends from the first film material 100. The second film material 110 may extend from a top side 104, the bottom side 102, or may be integrated into the first film material 100. The second film material 110 may be attached to the first film material 100 by way of adhesive, stitching, welding, a combination thereof, or the like. The second film material 110 may be sealed to the first film material 100. In some examples, the second film material 110 may be positioned between multiple layers of the first film material 100, may extend from a bottom side 102 of the first film material 100, or may be an extension of the same material as the first film material 100. In some examples the first film material 100 and the second film material 110 are the same material. In other examples the first film material 100 and the second film material 110 are different materials. In one specific example, the first film material 100 is a stiffer, or a more rigid material, than the second film material 110 and/or layering of a first film material 100 provides for a stiffer, or a more rigid material, than the second film material 110. In another example, the second film 110 material may be stiffer, or a more rigid material, than the first film material 100 and/or layering of a second film material 110 provides for a stiffer, or a more rigid material, than the first film material 100. In some examples, the first film material 100 and/or the second film material 110 and/or accompanying dressing may be a clear material for monitoring the wound.

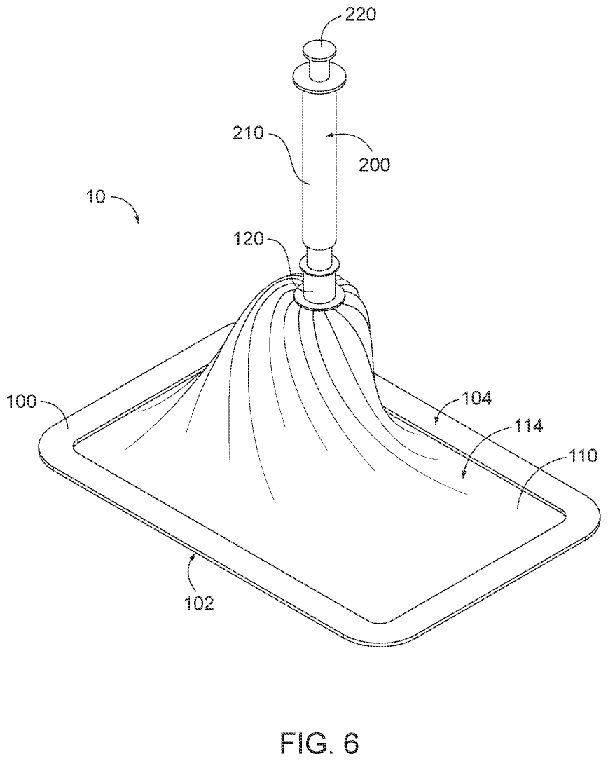

A valve 120 extends from the second film material 110. The valve 120 may be a self-sealing valve. An example of a self-sealing valve may be a one-way valve. The valve 120 is sealingly attached to the second film material 110. When the valve is in an open position an aperture, or passage, extends through the second film material 110 by way of the valve 120. In examples, the valve 120 is flush with the bottom side 112 (as illustrated by FIG. 3) of the second film material 110. In examples, the valve 120 does not extend through the bottom side 112 (as illustrated by FIG. 3) of the second film material 110 but, instead, may be flush with the bottom side 112 of the second film material 110. The valve 120 may extend from the top side 114 of the second film material 110. The valve 120 may comprise a receiver 125 at a top side 124 for receiving a removable medical syringe 200, as illustrated by FIG. 6. Moreover, the valve 120 may have threads 127 for receiving the removable medical syringe 200, as illustrated by FIGS. 6, 11, and 12A-12B. Other mechanisms for securing a removable medical syringe 200 to the valve 120 are contemplated herein and may include other fittings or connections known in the art. The mechanism for securing a removable medical syringe 200 may be a universal connection for connection to a standardized medical syringe readily available and utilized by medical professionals or within first aid kits.

In one example, the valve 120 is a one-way valve comprising a flap 128. The flap 128 is maintained in a closed position within the valve 120 unless air, fluid, or another device engages the flap 128 to move the flap 128 to an opened position. In one specific example, a flap 128 may be positioned within the opening, or passage 126, of the valve and extend from a closed position to an open position in a direction of the top side 124 of the valve 120. When the syringe 200 is positioned on the valve 120, the syringe 200 may be operated to pull air or fluid through the valve 120 from the bottom side 122 of the valve 120 to the top side 124 of the valve 120 where the flap 128 is moved from the closed position to the open position by way of the air or fluid movement. Upon ceasing operation of the syringe 200, or positioning of the syringe 200 within the valve 120, the flap 128 of the valve 120 returns to its closed position sealing the valve 120. Other examples of a self-sealing valve may include a flap 128 that extends across a top side 114 of the second film 110 material. The flap 128 may be moved in order to insert a syringe 200 into an opening through the second film material 110. Upon utilizing and removing the syringe 200, the flap 128 may be returned to across the opening of the second film material 110 sealing the opening in the second film material 110. Additional arrangements of the above self-sealing valves are contemplated herein. Other self-sealing valves are also contemplated herein to accomplish the purpose as set forth herein.

The first film material 100, the second film material 110, and the valve 120 form a contiguous vacuum barrier when the valve 120 is closed. In one example, each of the first film material 100, the second film material 110 and the valve 120, aside from the passage 126 through the valve 120 when in the open position, are of non-porous material. In some examples, only the second film material 110 and the valve 120 may form the contiguous vapor barrier and be of the non-porous material. Upon adhering the first film material 100 to the skin, or body, the contiguous vacuum barrier creates an impervious seal, aside from the valve opening when the valve is in an open position, over the wound onto which it is positioned. This allows the aforementioned removable medical syringe 200 to remove air or fluids from between the wound and the contiguous vacuum barrier, through the opening 126 of the valve 120 when the valve 120 is in an open position, providing negative pressure, or a vacuum, between the contiguous vacuum barrier and the wound. The second film material 110 of the contiguous vacuum barrier is pulled into the wound and applies pressure onto the wound by way of the negative pressure. Because the valve 120 is a self-sealing valve it seals upon ceasing operation of the removable medical syringe 200 or upon removal of the removable medical syringe from the valve 120. This negative pressure, or vacuum, is maintained between the second film material 110 until the material is cut or removed from the body, or skin. Unlike the VAC devices noted above, a vacuum pump is not needed for the vacuum sealed bandage of the present disclosure. Unlike the VAC devices noted above, a vacuum pump is not maintained in continuous operation on a closure to maintain the negative pressure or vacuum on a wound for the vacuum sealed bandage of the present disclosure. Unlike the VAC devices noted above, a power supply and/or an electro-mechanical device are not included with, and do not form a part of, the vacuum sealed bandage of the present disclosure. Unlike the VAC devices noted above, a tube is not positioned between the closure and the wound for drawing air or fluids from within the wound for a vacuum sealed bandage of the present disclosure. Unlike the VAC devices noted above, an independent tube extending through the closure is not provided with and does not form a part of the vacuum sealed bandage of the present disclosure. Additionally, or alternatively, the first film material 100 and the second film material 110 may also be a water tight material and/or application providing for showering or cleaning of a patient while still protecting the wound not otherwise possible with use of an electro-mechanical device and/or a power supply.

Because the vacuum sealed bandage 10 of the present disclosure has a valve 120, accessibility to a wound is maintained while maintaining the vacuum sealed bandage 10 over the wound. Specifically, the wound may be repeatedly drained, or reassessed, by way of a removable medical syringe 200 (as illustrated by FIG. 6) while the vacuum sealed bandage 10 remains in place. In other words, the vacuum sealed bandage 10 need not be removed for adjusting the negative pressure, or vacuum, between the second film material 110 and the wound and/or for removing additional air or fluids from between the second film material 110 and the wound. Moreover, if a clear second film material 110 and/or clear accompanying dressing is utilized, as noted above, the wound area may be continuously monitored for changes in condition, infection, an increase in fluid, or the like, for attention of the same. By maintaining the vacuum sealed bandage 10 on the wound, bleeding is contained and the risk for infection is also contained, or reduces the increase of infection. These benefits are imparted while also increasing mobility of the patient by not requiring an electro-mechanical device, such as a vacuum pump, and/or a power supply to maintain added or negative pressure on the wound.

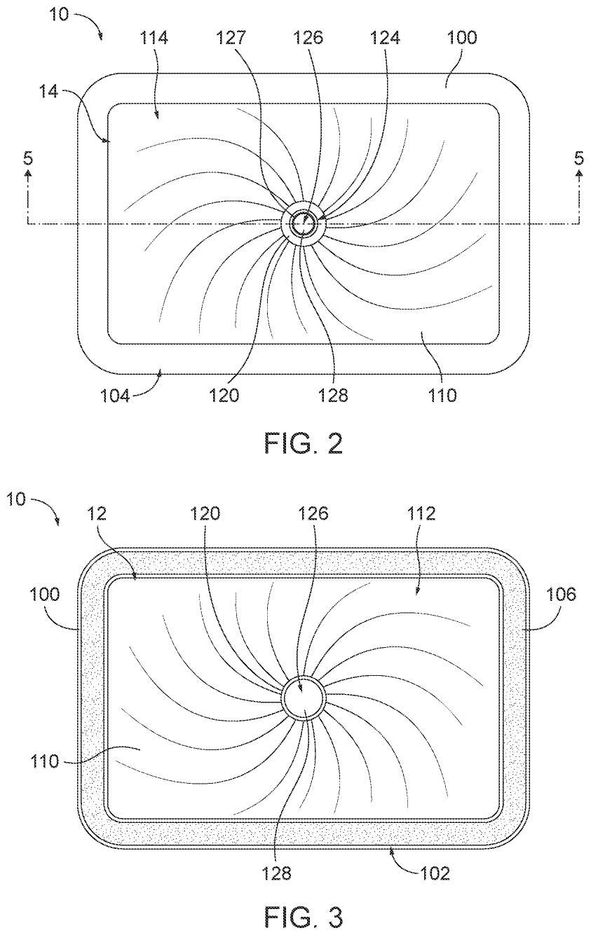

FIG. 2 is a top side 14 view of a vacuum sealed bandage 10 with features as described with respect to FIG. 1. In FIG. 2, the top side 104 of the first film material 100 is illustrated. The second film material 110 extends from the first film material 100 where the first film material 100 forms a perimeter about the second film material 110. A valve 120 extends from at top side 114 of the second film material 110 and is sealingly attached to the second film material 110. A passage 126 extends through the valve 120 from the top side 124 of the valve 120 through a bottom side 122 of the valve 120 (as illustrated by FIG. 3). A noted above, the valve 120 may be a self-sealing valve. FIG. 2 illustrates a flap 128 provided within the valve 120 to seal the valve 120 when air or fluid is not being pulled through the passage 126 of the valve 120, such as when a syringe 200 (as illustrated in FIG. 6) forces air or fluid through the valve as described herein. Otherwise, the flap 128 of the valve 120 is maintained in a closed position, thereby, closing and sealing the self-sealing valve. Additionally, or alternatively, a cap may be secured to the top side of the valve 120 to additionally, or alternatively, close or seal the valve 120 when the removable medical syringe 200 (as illustrated by FIG. 6) is not positioned within the valve 120. A cap may be secured to the valve 120 in the same manner as the removable medical syringe 200 (as illustrated by FIG. 6) such as, for example, threaded connection, adhesive connection, or other connections as known in the art.

Turning to FIG. 3, a bottom side 12 of a vacuum sealed bandage 10 is illustrated. As mentioned above, an adhesive 106 is provided on the bottom side 102 of the first film material 100. The adhesive 106 is configured to be applied to the skin, or body, to adhere the first film material 100 in an airtight arrangement, or sealed, with the skin, or body, of a patient. FIG. 3 also illustrates the bottom side 112 of the second film material 110 with the passage 126 of the valve 120 extending therethrough. As noted above, a flap 128 is positioned within the valve 120 in order to seal the valve 120 when air or fluids are not being transferred through the passage 126.

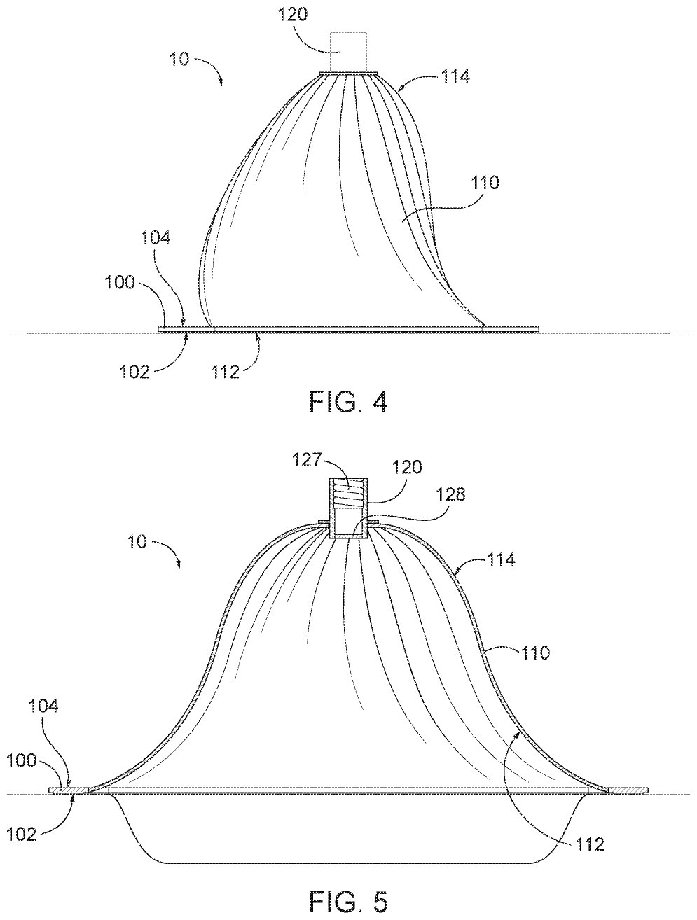

Turning now to FIG. 4, a side view of a vacuum sealed bandage 10 is illustrated. The bottom side 102 and the top side 104 of the first film material 100 is illustrated and is orientated in the same fashion as the bottom side 112 and the top side 114 of the second film material 110, as illustrated by FIGS. 3 and 2, respectively. The valve 120 is illustrated extending from the vacuum sealed bandage 10 in a direction of the respective top sides 104, 114. The valve 120 extends from a top side 114 of the second film material 110 but remains flush with the bottom side 112 of the second film material 110 so not to engage or impede engagement of the bottom side 112 of the second film material 110 and a wound as described herein, or as an added tube would do when utilizing a VAC device that extends entirely through the closure of the VAC device or positioned between the closure and the wound.

In one example, the second film material 110 is loosely arranged, or configured, relative the first film material 100. In other words, the second film material 110 may be loosely configured relative the first film material 100 such that it may be manipulated into multiple positions and arrangements, relative the first film material 100, and/or it may be moved from one side to another side, or to an opposite side (e.g., from the top side 104 to the bottom side 102), of the first film material 100. As illustrated here, the second film material 110 may be moved from the top side 104 of the first film material 100 to the bottom side 102 of the first film material 100 within the perimeter formed by the first film material 100. In examples, the second film material 110 may be folded so that it is positioned on the same plane as the first film material 100. The second film material 110 may be folded such as a parachute may be folded so that it unfolds, releases, and/or expands upon applying a negative pressure between the second film material 110 and a wound. In examples, the second film material 110 may be provided in a twisted arrangement extending from the top side 104 of the first film material 100 with the valve positioned to the top of the twisted arrangement. The second film material 110 may become untwisted, release, and/or expand through the perimeter of the first film material 100 into the wound upon applying a negative pressure between the second film material 110 and a wound. Generally, the loosely configured second film material 110 is provided for advancing the second film material 110 from the first film material 100 into a wound when applying negative pressure between the second film material 110 and the wound. In some examples, the loosely configured second film material 110 may extend as little as ½ inch from the first film material 100 or as much as 4 or 5 inches from the first film material 100.

Turning now to FIG. 5, the description of FIG. 4 is further illustrated by way of a cross-section of a vacuum sealed bandage 10 of the present disclosure, taken along line 5-5 of FIG. 2. The features of FIG. 5 are labeled as described with respect to FIG. 4 and are not repeated here for the sake of brevity.

In operation, a removable medical syringe 200 (as illustrated by FIG. 6) is attached to, or inserted into, the valve 120 of a vacuum sealed bandage 10 that is adhered and sealed to the skin, or body, of a patient. An example of a vacuum sealed bandage 10 with a removable medical syringe 200 is illustrated by FIG. 6 with features for the vacuum sealed bandage 10 as previously described with respect to FIG. 1. The features of FIG. 6 are labeled as described with respect to FIG. 1 and are not repeated here for the sake of brevity. In this example, the removable medical syringe 200 is sealably connected to the self-sealing valve 120. Operation of the removable medical syringe 200, such as pulling up on a slidable plunger 220 positioned within the tube 210 of the syringe 200, creates a pump action and a vacuum, or negative pressure, which pulls air or fluid into the tube 210 of the syringe 200. Air or fluids are pulled through the valve 120 from between the bottom side of the contiguous vacuum barrier, and more specifically the bottom side 112 of the second film material 110 (as illustrated by FIG. 3), and the wound over which the vacuum sealed bandage 10 is positioned. The pump action of the syringe 200, causing the vacuum, or negative pressure, draws the loosely configured second film material 110 in through the perimeter formed by the first film material 100 and into the wound, thereby, applying pressure into and/or onto the wound. As noted above, the vacuum, or negative pressure, may additionally unfold or untwist an otherwise folded or twisted second film material 110 such as a parachute unfolds or untwists when having positive pressure applied to one side of the parachute and opposing negative pressure, or a different pressure, applied to the opposite side of the parachute. Additionally, or alternatively, the operation of the syringe 200 may also adjust the amount of negative pressure between a wound and the vacuum barrier by applying more negative pressure between a wound and the vacuum barrier or reversing course and increase pressure between a wound and the vacuum barrier.

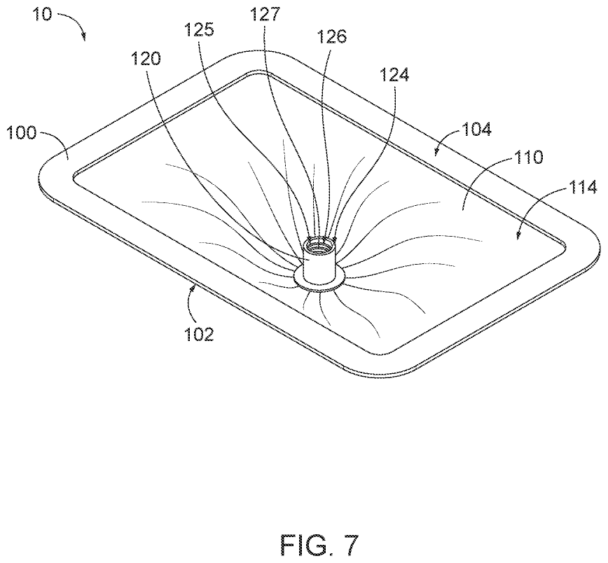

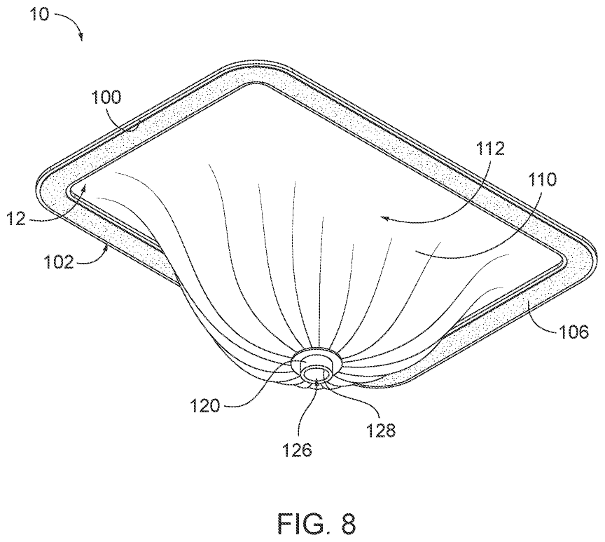

This progression of the second film material 110 is best illustrated by FIGS. 7-8. In FIG. 7, a top side perspective view illustrates a second film material 110 of a vacuum sealed bandage 10 as it becomes untwisted from a twisted arrangement as illustrated by FIG. 1. The features of the vacuum sealed bandage 10 of FIG. 7 are labeled as described with respect to FIG. 1 and are not repeated here for the sake of brevity. FIG. 8 illustrates a bottom side perspective view of a second film material 110 of a vacuum sealed bandage 10 after it has been pulled through the perimeter of the first film material 100 in a direction of the bottom side 102 of the first film material 100 and into a wound. The features of FIG. 8 are labeled as described with respect to FIG. 3 and are not repeated here for the sake of brevity.

In operation, the vacuum, or negative pressure, imparted by the syringe manipulates the second film material into the wound to tamponade bleeding of the wound. Bandage or dressing materials, such as gauze or sponges, may also be provided between the second film material and the wound forcing the additional bandage material into the wound. In other words, the vacuum sealed bandage may be used in combination with added packing for a wound. Bandage or dressing materials, such as gauze or sponges, may also be attached or adhered to the bottom side of the second film material. In some examples, the second film material may be forced directly into the wound and adhere to the wound. In addition to bandage or dressing materials, antimicrobial treatments may be further applied to components of the vacuum sealed bandage or the dressing materials accompanying the vacuum sealed bandage. In examples, the vacuum, or negative pressure, may drive the bandage materials and/or the second film material to tunnel into the wound, or force bandage material, gauze, or sponges to tunnel into the wound, to a wound area that is not otherwise exposed at the surface of the wound or skin area or that is otherwise accessible from the surface of the wound or skin area. In some examples, the second film material may tunnel into a wound as much as ½ inch and as much as 4 or 5 inches. Thereby, the vacuum sealed bandage provides additional pressure to the wound in such a tunneled arrangement that is not otherwise achieved by applying a material directly to the surface of the wound.

The vacuum sealed bandage of the present disclosure maintains negative pressure between the contiguous vacuum barrier, or the second film material, and the wound even upon removal of the removable medical syringe or upon ceasing operation of the removable medical syringe. Because the negative pressure is maintained, the vacuum sealed bandage operates without continuous use of a electro-mechanical device, such as a vacuum pump, or without requiring a power supply. More specifically, the vacuum sealed bandage does not have, or require, an electro-mechanical device, such as a vacuum pump, or require a power supply that accompanies such devices. Additionally, because the vacuum sealed bandage of the present disclosure has an integral valve additional tubes, for transfer of air or fluids from between a closure and a wound, are additionally not present or required. In other words, the vacuum sealed bandage of the present disclosure does not have, or require, added or inserted tubes for transferring air or fluids from between the vacuum barrier and the wound.

A method of applying a vacuum sealed bandage to a wound is also disclosed herein. In one example, the method comprises adhering a first film material having an adhesive on one side to a skin surface, or body of a patient. The first film material is sealed to the skin surface, or body of a patient, forming an airtight arrangement with the skin surface, or body of a patient. A removable medical syringe is sealingly attached to a self-sealing valve. Sealingly attaching the removable medical syringe to the self-sealing valve may include threading the removable medical syringe to the self-sealing valve. The self-sealing valve extends from a top side of a second film material and is sealingly attached to the second film material. The second film material extends from the first film material where one or more of the first film material, the second film material, and the self-sealing valve form a contiguous vacuum barrier when the self-sealing valve is in a closed position. Negative pressure is created between the second film material and a wound in the skin by drawing air or fluid from between the contiguous vacuum barrier and the wound into the removable medical syringe. The second film material is thereby manipulated into the wound by way of drawing air or fluid from between the contiguous vacuum barrier and the wound. Finally, the removable medical syringe may be removed from the valve where the valve is a self-sealing valve and seals upon removal of the removable medical syringe. The method may further comprise maintaining negative pressure created between the second film material and the wound upon removing the removable medical syringe.

Methods of applying a vacuum sealed bandage to a wound may additionally comprise unfolding or untwisting the second film material when manipulating the second film material into the wound. The second film material may be folded or twisted in a stored position before the step of adhering. When manipulating the second film material into the wound, the second film material may be moved from one side of the first film material to the opposite side of the first film material. More specifically, the second film material may be moved from one side of the first film material to the opposite side of the first film material having the adhesive. The method may also comprise adjusting the amount of negative pressure between the second film material and a wound by adjusting the removable medical syringe. Moreover, the second film material may be tunneled into the wound.

As noted above, the operation, or methods of applying a vacuum sealed bandage to a wound are performed without electro-mechanical devices, or a vacuum pump, and/or without a power supply as required for such devices. Additionally, the operation, or methods of applying a vacuum sealed bandage to a wound are performed without an inserted tube extending from one side of the vacuum barrier to the opposite side of the vacuum barrier.

The method of applying the vacuum sealed bandage to a wound may further comprise adhering a first film material of a second vacuum sealed bandage to a skin surface, or body of a patient. The second vacuum sealed bandage is larger than and positioned overtop the first vacuum sealed bandage. The first film material of the second vacuum sealed bandage also has an adhesive on one side forming an airtight, or sealed, arrangement with the skin surface, or body of a patient. After performing the above steps on the first vacuum sealed bandage, the second vacuum sealed bandage may have a removable medical syringe sealingly attached to a self-sealing valve. The self-sealing valve extends from, and is sealingly attached to, the second film material of the second vacuum bandage where the second film material of the second vacuum sealed bandage extends from the first film material of the second vacuum sealed bandage. At least one or more of the first film material, the second film material, and the self-sealing valve of the second sealed bandage form a contiguous vacuum barrier when the self-sealing valve is in a closed position. Negative pressure may be created or provided between the second film material of the second vacuum sealed bandage and a first vacuum sealed bandage by drawing air or fluid from between the contiguous vacuum barrier of the second vacuum sealed bandage and the first vacuum sealed bandage into the removable medical syringe. The second film material of the second vacuum sealed bandage may be manipulated into the first vacuum sealed bandage and the wound by way of drawing air and fluid from between the contiguous vacuum barrier of the second sealed bandage and the first vacuum sealed bandage. Finally, the removable medical syringe may cease operation and/or be removed from the self-sealing valve of the second vacuum sealed bandage where the self-sealing valve seals upon ceasing operation of or removing the removable medical syringe. Additional layers of vacuum sealed bandages may be applied as described by applying a second vacuum sealed bandage to a first vacuum sealed bandage above.

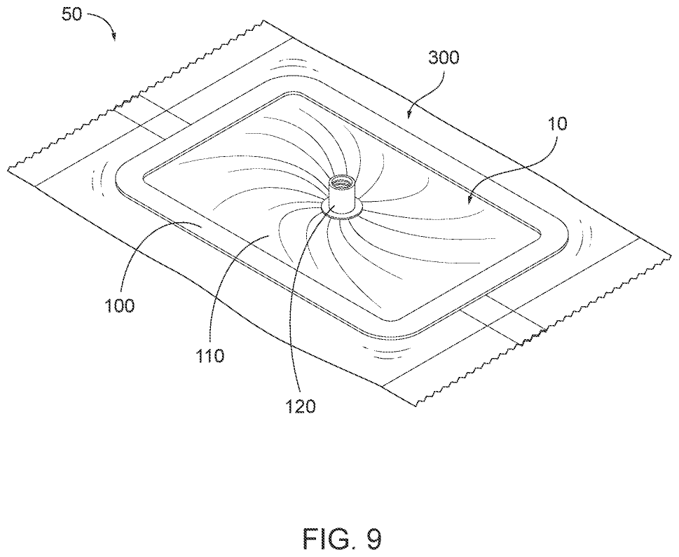

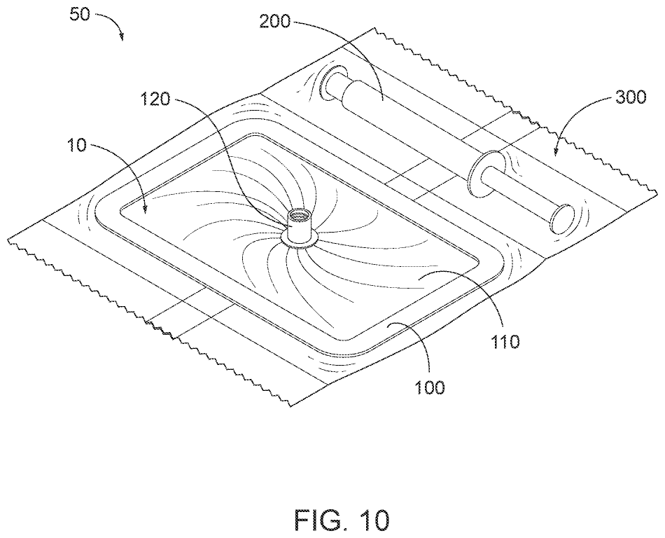

Turning now to FIGS. 9-10, an example of a packaged vacuum sealed bandage system 50 is illustrated. The vacuum sealed bandage system may comprise a sterile wrapping 300. A vacuum sealed bandage 10 may be sealed within the sterile wrapping 300. The vacuum sealed bandage 10 may have a first film material 100 having adhesive on one side where that side is configured to be applied to skin, or a body, to adhere the first film material 100 in a sealed airtight arrangement with the skin. A second film material 110 extends from and is sealingly secured to the first film material 100. A self-sealing valve 120 extends from and sealingly attaches to the second film material 110. At least one of the first film material 100, the second film material 110, and the self-sealing valve 120 form a contiguous vacuum barrier when the self-sealing valve is closed. Further, the second film material 110 may be manipulable into multiple positions or arrangements such as, for example, the second film material 110 may be in a folded arrangement within the sterile wrapping 300. Additionally, or alternatively, the second film material 110 may be in a twisted arrangement within the sterile wrapping 300. The vacuum sealed bandage system 50 may further comprise a removable medical syringe 200, as illustrated by FIG. 10, additionally sealed within the sterile wrapping 300. As emphasized above, such systems are without an added tube, without an electro-mechanical device, such as a vacuum pump, and are without requiring a power supply and, stated differently are not for use with the same. Because such a system is affirmatively absent these particular features of the above packaged vacuum sealed bandage systems 50 may be stated, or claimed, as “consisting of” or “consist essentially of” any combination or arrangement of the features illustrated by and described with respect to FIG. 9 or 10.

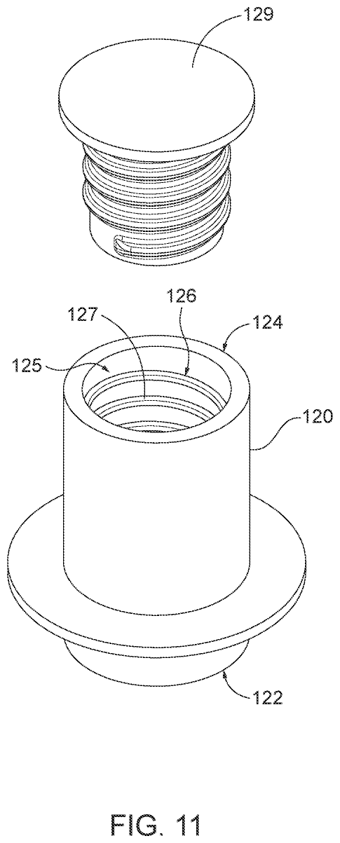

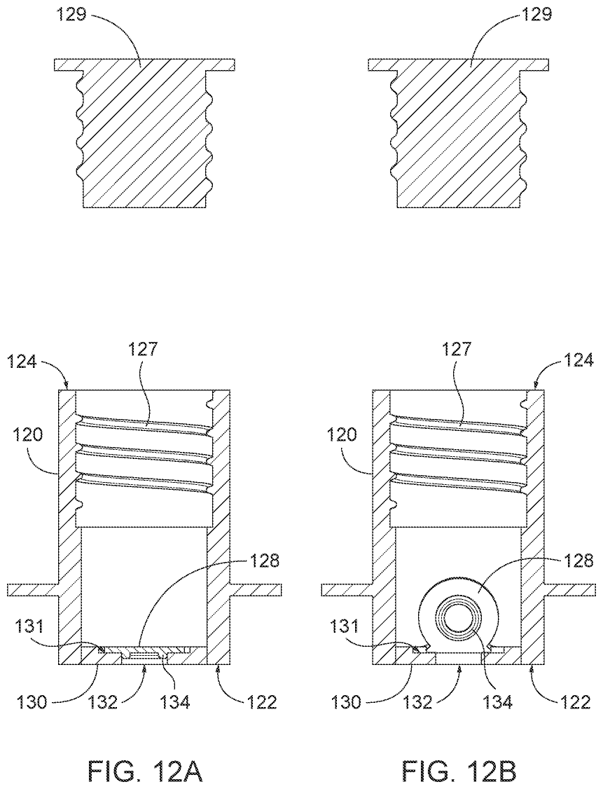

FIG. 11 illustrates an example of a self-sealing valve 120 of the present disclosure. In this example, the valve 120 is a one-way valve comprising a flap 128 (as illustrated by FIGS. 12A and 12B). The flap 128 (as illustrated by FIGS. 12A and 12B) is maintained in a closed position within the valve 120 unless air, fluid, or another device engages or forces the flap 128 to move to an opened position. The flap, otherwise, maintains sufficient rigidity, in combination with vacuum pressure, to be maintained in a closed position. A spring arrangement may additionally, or alternatively, be relied on to maintain or force the flap to a closed position. In one specific example, a flap 128 may be positioned within the opening, or passage 126, of the valve and extend from a closed position to an open position in a direction of the top side 124 of the valve 120. When the syringe is positioned within a receiver 125 of the valve 120, the syringe may be operated to pull air or fluid through the valve 120 from the bottom side 122 of the valve 120 to the top side 124 of the valve 120 where the flap 128 is moved from the closed position to the open position by way of the air or fluid movement. Upon ceasing operation of the syringe, or positioning of the syringe within the valve 120, the flap 128 of the valve 120 returns to its closed position sealing the valve 120. Other examples of a self-sealing valve may include a flap 128 that extends across a top side of the second film material. The flap 128 may be moved in order to insert a syringe into an opening through the second film material. Upon utilizing and removing the syringe, the flap 128 may be returned to across the opening of the second film material sealing the opening in the second film material. The valve 120 may have threads 127 for receiving the removable medical syringe 200, as illustrated by FIGS. 6, 11, and 12A-12B. In these examples, the threads 127 of the valve 120 are on the inside of the valve 120. However, it may additionally be contemplated where the threads of the valve are additionally, or alternatively, on the exterior of the valve to receive a syringe of a different kind or a combination of syringes or devices. FIG. 11 also illustrates a cap 129 that may be utilized to further seal the valve 120 once a syringe is removed. The cap 129 may be threaded for mating with the threads 127 of the valve 120. The cap 129 may be made of a rubber material so to create a seal with the valve. Other materials, such as, for example, other polymers, are also contemplated herein. Further, an O-ring or other sealing devices may accompany the cap 129 for additionally sealing the valve. Additional arrangements of the above self-sealing valves are contemplated herein. Other self-sealing valves are also contemplated herein to accomplish the purpose as set forth herein.

Turning now to FIGS. 12A-12B, cross-sections of the valve 120 of FIG. 11 are illustrated. In FIGS. 12A-12B, the valve 120 is illustrated with threads 127 at the top side for receiving a syringe. A flap 128 is illustrated at the bottom side. Other arrangements, such as, for example, providing the flap centrally within the valve or at the top side of the valve are also contemplated herein. In FIG. 12A, the valve 120 is illustrated as being closed. The flap 128 is seated on a ledge 130 having an aperture 132 extending therethrough. The aperture 132 is an extension of the passage 126 of the valve when the valve 120 is opened (as illustrated in FIG. 12B). The flap 128 comprises a curb 134, or raised edges, for seating against the ledge 130 within the aperture 132. The ledge 130 may also comprise a recess 131 for receiving the flap 128. By seating the flap 127 on the recess 131 and extending the curb 134 of the flap into the aperture and biasing the ledge 132 a seal is formed between the curb 134 and the ledge 130 and/or the flap 128 and the recess 131 for forming an airtight seal. FIG. 12B illustrates the valve 120 in an open arrangement where the flap 128 is in a raised position as air, or fluid, is transferred through the passage 126. The curb 134 is illustrated to the bottom side of the flap 128 for mating with the ledge 130. In this example, the flap is secured to one side of the ledge 130 such that the flap may otherwise flip up to the open position and flip down to the closed position while being secured to the valve arrangement, or ledge 130. The flap 128 may be made of a rubber material so to create a seal with the ledge 130 while maintaining flexibility to raise and lower while being secured at one end to the ledge 130. Other materials, such as, for example, other polymers, are also contemplated herein. A hinge may also be relied on for raising and lowering said flap 128. Also illustrated by FIGS. 12A and 12B is the cap 129 as described above with respect to FIG. 11.

While this invention has been described with reference to examples thereof, it shall be understood that such description is by way of illustration only and should not be construed as limiting the scope of the claimed examples. Accordingly, the scope and content of the examples are to be defined only by the terms of the following claims. Furthermore, it is understood that the features of any example discussed herein may be combined with one or more features of any one or more examples otherwise discussed or contemplated herein unless otherwise stated.

What is claimed is:

1. A vacuum sealed bandage comprising:
a first film material having adhesive on one side configured to be applied to skin and to adhere the first film material in an airtight arrangement with the skin;
a second film material extending from the first film material; and
a self-sealing valve extending from and sealingly attached to the second film material;
where the first film material, the second film material, and the self-sealing valve form a contiguous vacuum barrier when the self-sealing valve is closed and the second film material is manipulable into multiple positions or arrangements.

2. The vacuum sealed bandage of claim 1 where the first film material forms a perimeter about the second film material.

3. The vacuum sealed bandage of claim 2 where the second film material is loosely configured to be moved from one side of the first film material to the opposite side of the first film material through the perimeter formed by the first film material.

4. The vacuum sealed bandage of claim 1 where the self-sealing valve extends from a first side of the vacuum barrier and is flush on a second side of the vacuum barrier.

5. The vacuum sealed bandage of claim 4 where the second film material extends from a side of the first film material in the same direction as the self-sealing valve extends from the first side of the vacuum barrier.

6. The vacuum sealed bandage of claim 1 where the second film material is folded upon itself.

7. The vacuum sealed bandage of claim 1 where the first film material is more rigid than the second film material.

8. The vacuum sealed bandage of claim 1 where the first film material is layered and the second film material extends from between the layers of the first film material.

9. The vacuum sealed bandage of claim 1 having a removable medical syringe inserted into the self-sealing valve holding the self-sealing valve open.

10. The vacuum sealed bandage of claim 9 where operation of the removable medical syringe creates negative pressure between a wound and the vacuum barrier when the first film material is in the airtight arrangement with the skin.

11. The vacuum sealed bandage of claim 10 where the removable medical syringe adjusts the amount of negative pressure between a wound and the vacuum barrier.

12. The vacuum sealed bandage of claim 10 where the negative pressure manipulates the second film material into the wound.

13. The vacuum sealed bandage of claim 10 where negative pressure is maintained between the second film material and the wound without a vacuum pump or does not require a power supply for operation.

14. The vacuum sealed bandage of claim 1 that does not have a vacuum pump or does not require a power supply for operation.

15. A method of applying a vacuum sealed bandage to a wound, the method comprising the steps of:
adhering a first film material having an adhesive on one side to a skin surface forming an airtight arrangement with the skin surface;
sealingly attaching a removable medical syringe to a self-sealing valve where the self-sealing valve is sealingly attached to a second film material that extends from the first film material where the first film material, the second film material, and the self-sealing valve form a contiguous vacuum barrier when the self-sealing valve is closed;
creating negative pressure between the second film material and a wound in the skin by drawing air or fluid from between the contiguous vacuum barrier and the wound into the removable medical syringe;
manipulating the second film material into the wound by way of drawing air or fluid from between the contiguous vacuum barrier and the wound; and
removing the removable medical syringe from the self-sealing valve where the self-sealing valve seals upon removal of the removable medical syringe.

16. The method of applying the vacuum sealed bandage to the wound of claim 15, the method further comprising the step of:
untwisting the second film material when manipulating the second film material into the wound wherein the second film material is in a twisted arrangement before the step of adhering.

17. The method of applying the vacuum sealed bandage to the wound of claim 15, the method further comprising the step of:
moving the second film material from one side of the first film material to the opposite side of the first film material having the adhesive when manipulating the second film material into the wound.

18. The method of applying the vacuum sealed bandage to the wound of claim 15, the method further comprising the step of:
maintaining the negative pressure created between the second film material and the wound upon removing the removable medical syringe.

19. The method of applying the vacuum sealed bandage to the wound of claim 15, the method further comprising the step of:
tunneling the second film material into the wound.

20. The method of applying the vacuum sealed bandage to the wound of claim 15 performed without a vacuum pump or without a power supply.
